(19) United States
(12) Patent Application Publication
(10) Pub. No.: US 2024/0338497 A1
(43) Pub. Date: Oct. 10, 2024

(54) SYSTEM AND METHOD FOR A DIGITAL HEALTH PLATFORM PROVIDING REMOTE RESIDENCE HAZARD ANALYSIS

(71) Applicant: RenovaHealth Corp., Boston, MA (US)

(72) Inventor: Gracyn Robinson, Boston, MA (US)

(21) Appl. No.: 18/629,170

(22) Filed: Apr. 8, 2024

Related U.S. Application Data
(60) Provisional application No. 63/457,759, filed on Apr. 6, 2023.

(51) Int. Cl. G06F 30/13; G16H 40/67

(52) U.S. Cl. CPC G06F 30/13; G16H 40/67

(57) ABSTRACT

Systems and methods for providing a digital health platform that provides remote residence hazard analysis and remediation are discussed.

### RELATED APPLICATION

This application claims priority to, and the benefit of, U.S. Provisional Patent Application No. 63/457,759, filed Apr. 6, 2023, entitled “Digital Health Platform”, the contents of which are incorporated herein by reference in their entirety.

### BACKGROUND

One out of three people over the age of 65 will experience a fall within their home. Some of these falls cause fatalities but even the non-fatal falls cause a variety of issues such as bruises, strains, and broken bones and with each fall the likelihood of successive falls increases greatly. These falls may result in lasting trauma that threatens the health and safety of seniors as well as their economic security and independence. Falls within the home lead to millions of injuries and hundreds of thousands of hospitalizations each year. The falls also result in billions of dollars in cost burdens upon society, a number that is only expected to increase over the coming decades.

In recent years, a number of different technologies have been developed to create 3D graphical views from captured scan or image data of a location of interest. These 3D graphical views enable immersive guided tours around the captured environment and are frequently used for example for virtual tours of museums and historical sites and by realtors for virtual tours of properties that are being listed for sale.

### BRIEF SUMMARY

Embodiments of the present invention provide an online digital health platform to assist seniors in identifying potential hazards in their residence and provides potential remedies to re-configure and/or renovate portions of their residences to address the identified hazards. More particularly, embodiments enable a resident to interact with the digital health platform (hereafter “health platform”) either through an online portal or via a downloaded app. The health platform receives residence information that includes information about a residence and at least one resident of the residence. The health platform utilizes the residence information and a 3D graphical model generated from scan data or image data captured from the residence to identify potential hazards. In some embodiments, an augmented reality (AR) view of portions of the residence is displayed in which hotspot indicators indicating potential hazards are inserted and displayed within the captured 3D graphical view of the residence. In some embodiments, graphical indicators and/or graphical depictions of suggested re-configurations/renovations to address the identified hazards are displayed within this 3D AR view.

In one embodiment, a computing-device implemented method for providing a digital health platform via one or more computing devices that are each equipped with one or more processors includes receiving residence data over a network. The residence data is data associated with a residence and at least one resident of the residence. The method further includes identifying one or more areas of hazard in a 3D graphical model created from captured data of the residence. The captured data is one or more of scan or image data taken of the residence. The method additionally includes creating a 3D Augmented Reality (AR) model by inserting an indicator for each of the one or more identified areas of hazard into the 3D graphical model and inserting one or more graphical indicators of suggested re-configurations/renovations of the residence into the 3D AR model. The method further includes providing a display of the 3D AR model with the suggested re-configurations/renovations of the residence.

In another embodiment, a computing-device implemented method for providing a digital health platform via one or more computing devices that are each equipped with one or more processors includes receiving over a network residence data. The residence data is data associated with a residence and at least one resident of the residence. The method further includes identifying one or more areas of hazard in a graphical model created from captured data. The captured data is one or more of scan data or image data taken of the residence. The method additionally includes inserting an indicator for each of the one or more identified areas of hazard into the graphical model and inserting one or more graphical indicators of suggested re-configurations/renovations of the residence into the graphical model. The method also includes providing a display of the graphical model with the suggested re-configurations/renovations of the residence.

In one embodiment, a system for a digital health platform provides remote residence hazard analysis and includes one or more computing devices equipped with one or more processors configured to provide a network-accessible digital health platform. The digital health platform includes a network interface configured to receive residence data. The residence data is data associated with a residence and at least one resident of the residence. The digital health platform further includes a hotspot module that is configured to identify one or more areas of hazard in a 3D graphical model created from captured data. The captured data is one or more of scan data or image data of the residence. The hotspot module is further configured to create a 3D Augmented Reality (AR) model by inserting an indicator for each of the one or more identified areas of hazard into the 3D graphical model and insert one or more graphical indicators of suggested re-configurations/renovations of the residence into the 3D AR model. The digital health platform also includes one or more of a network portal providing access to a display of the 3D AR model with the suggested re-configurations/renovations of the residence and a downloadable app configured to provide a display of the 3D AR model with the suggested re-configurations/renovations of the residence.

### BRIEF DESCRIPTION OF THE DRAWINGS

The accompanying drawings, which are incorporated in and constitute a part of this specification, illustrate one or more embodiments of the invention and, together with the description, help to explain the invention. In the drawings:

FIG. 1 depicts an exemplary environment for a digital health platform in an embodiment;

FIG. 2A depicts an exemplary sequence of steps performed for identifying and proposing remedies for hazards in a residence in an exemplary embodiment utilizing residence information and a 3D graphical model created from captured data of the residence;

FIG. 2B depicts an exemplary sequence of steps performed for identifying and proposing remedies for hazards in a residence in an exemplary embodiment utilizing residence information and a graphical model created from captured data of the residence;

FIG. 3 depicts exemplary measurements in a 3D graphical model examined to identify potential hazards in a residence in an exemplary embodiment;

FIGS. 4A-4C depict 3D AR models of a residence that include inserted graphical hotspot indicators of potential hazards and associated displayed graphical indicators of suggested re-configurations/renovations of the residence in exemplary embodiments;

FIG. 5 depicts displays of suggested re-configurations/renovations integrated into a 3D graphical model of a bathroom in an exemplary embodiment;

FIG. 6 depicts before and after renderings of renovations suggested by the health platform displayed by a mobile app in an exemplary embodiment;

FIG. 7 depicts captured environments displayed by a mobile app in 2D and 3D form in an exemplary embodiment; and

FIG. 8 depicts a displayed dashboard of suggestions in an exemplary embodiment.

### DETAILED DESCRIPTION

Embodiments of the present invention provide immersive visual and spatial computing tools that combine the visual results of a hazard analysis designed to identify fall, mobility, wayfinding and other hazards in a residence occupied by older residents with suggested reconfigurations/renovations to address those hazards. The suggested reconfigurations and renovations may be linked to activities of daily living and desired outcomes for the residents. The health platform described herein further enables access to physical, cognitive, and social support services to allow the resident to age in place with dignity and independence.

Every year, 1 in 3 adults age 65 and over fall and many of these falls occur at home. These falls frequently result in injury. The monetary cost in the form of Medicaid payments, private insurance payments and out of pocket payments by the injured parties for these falls runs into the billions of dollars. Further such falls may make it difficult for an elderly individual to stay in their home afterwards, whether for fear of additional injury or the effects of the initial fall. There is currently a shortage of assisted living housing and nursing homes that is expected to get worse in the coming years, and injuries causing seniors to vacate their home exacerbate the problem. Embodiments of the present invention attempt to provide a solution to this assisted living/nursing home shortage by providing a technical solution for identifying hazards in the dwellings of seniors and proposing suggestions for renovating/reconfiguring those spaces to prevent such falls from occurring, thereby enabling older residents to age in place with a greater degree of safety and confidence.

Embodiments provide a digital health platform that utilizes augmented reality (AR) and, in some embodiments, artificial intelligence (AI) and generative (AI) to provide a personalized 3D visualization of a residence to identify and address potential hazards within homes. The analysis of potential hazards is customized not only based on the physical attributes of the residence, but also based on the physical and cognitive status of the resident. The health platform layers the display of captured spaces with inserted visible indicators of design hotspots that identify hazards and present possible solutions. For example, the hotspots may be embedded with information pairing activities of daily living (ADLs) with desired outcomes (DOs), while offering links to accessible design products, links to International Code Council codes, standards and dimensions, and resources including potential contractors to perform the work. Through leveraging AR and optionally the use of AI, the health platform enhances the well-being and safety of older adults and promotes a well-balanced environment conducive to healthy aging.

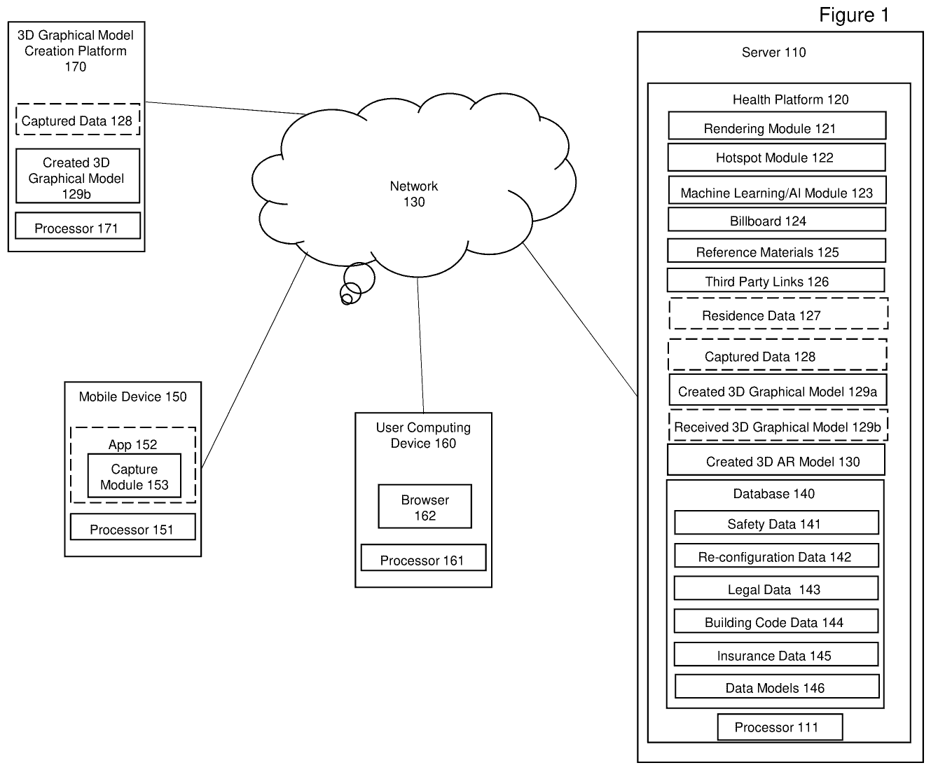

FIG. 1 depicts an exemplary environment for a digital health platform in an embodiment. Server 110 uses one or more processor(s) 111 to execute code to provide health platform 120. For example, processor(s) 111 may include a central processing unit (CPU), a graphical processing unit (GPU) or both. Server 110 may communicate over network 130 with mobile device 150 or user comping device 160 to receive and transmit information. Network 130 may be the Internet, an intranet, a cellular network or some other type of network enabling communication between server 110 and mobile device 150 or user computing device 160. Mobile device 150 and/or user computing device 160 are each equipped with one or more processors 151, 161. Mobile device 150 may be a smartphone, tablet, laptop or other mobile computing device. User computing device 160 may be a non-mobile computing device, such as, but not limited to, a desktop computer. In some embodiments, mobile device 150 and/or user computing device 160 may download app 152 which is designed to communicate with health platform 120. In some embodiments, app 152 may be downloaded from health platform 120. In other embodiments, app 152 may be retrieved from a different location such as a 3^{rd} party app store.

Health platform 120 is configured to receive residence data 127 associated with a residence and at least one resident of the residence over network 130. For example, residence data 127 may include information about the elderly resident such as particular health conditions, health and/or home insurance coverage, activities of daily living and age while the information associated with the residence may indicate the age and type of the structure and/or the number of occupants. It will be appreciated that additional types of data associated with the resident and the home environment are also within the scope of the present invention. Examples of types of residence data 127 include but are not limited to the following: residence dimensions, standard and/or regulated architectural clearances and spatial dimension tolerances, data regarding Activities of Daily Living and Desired Health Outcomes, room typical data within senior living community dwellings (sometimes referred to as “typicals” on architectural drawings), residence slope data indicating exterior ground slope, lighting levels, data regarding one or more paths of common travel within the home, as well between exits, means of egress, and/or data from generative AI data capture. In one embodiment this information may be transmitted to health platform 120 by a resident of the residence or someone on their behalf via app 152 from mobile device 150 or user computing device 160. In another embodiment, the resident or someone on their behalf may use mobile device 150 or user computing device 160 to log on to an online portal provided via a web page or other means by health platform 120 to provide the information.

In one embodiment, health platform 120 is configured to receive a 3D graphical model 129b created from captured data 128 where the 3D graphical model depicts some or all of the residence and/or its exterior. Captured data 128 is scan or image data of some or all of the rooms in a residence, and/or the exterior of the residence, that is suitable for creating an immersive graphical 3D view of the residence. In another embodiment, health platform 120 may also be configured to receive captured data 128 associated with the residence and create a 3D graphical model 129a of some or all of the residence and/or its exterior with the aid of rendering module 121 which assembles the captured data into the 3D graphical model. As used herein, the term ‘module’ refers to a circuit, a device, an electrical component, firmware and/or software for performing a task and accordingly it should be appreciated that the steps described herein as performed by a ‘module’ may be implemented via hardware, software or a combination thereof.

The captured data of a residence may be initially acquired in a number of ways. As non-limiting examples, the captured data may be scan data. It will be appreciated that the scan data may be acquired using a number of different techniques such as by gathering point cloud data, acquiring heat-mapping data, acquiring laser scanning data such as but not limited to LIDAR data, gathering radar capture data, acquiring ultrasound data, or acquiring data using other scanning techniques known in the art that are used to gather scan data that can be used to generate an accurate 3D graphical model of the environment. In one embodiment, the scan data may be captured using mobile device 150 (or the personal device of someone working on the resident's behalf) which may include app 152 and capture module 153 which controls available hardware and/software resources on the mobile device such as scan and image capabilities to acquire captured data 128. For example, recent versions of the iPhone® and iPad® have included LIDAR capabilities. In some embodiments, LIDAR data is acquired using time of flight principles to measure the time taken to receive reflections of transmitted laser signals thus capturing and measuring the locations of objects within an environment. In one embodiment, the use of a tripod during 3D partial scans diminishes the seams at the edges of the scans enabling them to be read as one, singular 360 degree image. In another embodiment, captured data 128 may be captured using a dedicated scanning device other than mobile device 150, such as, but not limited to, a separate LIDAR camera/sensor.

In another embodiment, the data may be image data captured using a stand-alone, high definition camera, or a camera integrated with mobile device 150. For example techniques such as photogrammetry may be used to assemble 3D graphical models from image data.

In one embodiment captured data 128 may include both data that is captured via scan and camera resources and data from generative AI. Generative AI data may include but is not limited to algorithmic data captured within the space (of which the output time speed will increase with repetitive use and calculations), item identification data (the output time speed of which will increase with repetitive use, calculations, and the use of machine learning), and resource information data (for which the input and output time speed of which will increase with use).

In one embodiment, app 152 is configured to send the collected scan or image data to health platform 120 to create 3D graphical model 129a. In another embodiment, the data may be separately uploaded to health platform 120 without the use of app 152. In one embodiment, the captured data may be streamed to health platform 120 for real-time analysis. For example, a resident may capture data with their phone, stream it to health platform 120 for analysis and receive hazard identification and suggestions for dealing with the same in real-time/near real-time.

In one embodiment, instead of health platform creating 3D graphical model 129a, a 3D graphical model 129b is separately created and transmitted to, and received by, health platform 120 for its use in analyzing the residence. For example, a third-party graphical model creation platform 170 which includes processor 171 may acquire and utilize captured data 128 to create 3D graphical model 129b and transmit it to health platform 120 over network 130.

Health platform 120 may include a number of software and/or hardware modules for specific tasks. For example, in one embodiment rendering module 121 uses captured data 128 to generate 3D graphical model 129a of the resident's home. In one embodiment, rendering module 121 is a BIMx rendering module and 3D graphical models 129a are BIMx renderings.

Health platform 120 includes hotspot module 122. Hotspot module 122 uses the 3D graphical model 129a, 129b in combination with the residence data and pre-defined criteria regarding hazards to identify potential hazards in the resident's living environment and perform a home safety analysis that attempts to identify potential hazards in a residence. The pre-defined criteria includes both code requirements and specified best practices. It should be appreciated that ‘hazard’ as used herein may refer not only to a dangerous condition that is of danger to the resident but also to an object or condition that may represent an impediment to continued use of the residence by an older adult even if not dangerous per se. As non-limiting examples, hazards may include fall dangers, wayfinding dangers (where a resident's path of foot travel from specific point A to point B may be dangerous for one reason or another or cause injury) and mobility hazards (where an object or condition in the residence presents a hazard to the resident either because of a characteristic of the condition or object alone or in combination with a current physical or mental condition of the resident).

For example, the citeria for hazard detection may include the following non-limiting list of considerations:
1. Are entrances protected from weather (e.g., canopies, walk-off mat)?
2. Does a room layout have clear and unobstructed paths of travel (e.g., storage, dedicated locations for commonly used moveable items, i.e. walker?).
3. Is the design location of the call button/systems accessible and usable by the resident?
4. Is the room layout designed so that the bathroom door is clearly identifiable from the bed?
5. Is the bathroom near the bed?
6. Is space provided on the opening side (door handle side) of the resident's toilet room door to facilitate the use of equipment and/or assistive devices?
7. Are smooth transitions allowed in walking surfaces or between flooring types to avoid surface irregularities leading to trips?
8. Is flooring designed with minimized glaring (e.g., flooring material, lighting, windows)?
9. What is the current design contrast to differentiate between the floors and walls?
10. Do the floor materials and patterning accurately convey the actual floor conditions (e.g., the perception of a level floor vs. a step or stair)?
11. Is there slip-resistant flooring in potential wet areas (e.g., bathrooms, entrances, kitchens) and on ramps and stairs?
12. Do flooring and subflooring materials mitigate injury in the event of a fall?
13. Are secure walk-off mats, rugs and carpeting to the floor present (e.g., entrances, lobbies, waiting areas)?
14. Is there lighting to eliminate abrupt changes in light levels causing shifts in visual acuity?
15. Is there low-level lighting in nighttime/dark conditions?
16. Are grab bars and handrails located to support residents while ambulating to the toilet?
17. Are grab bars located on either side of the toilet to support residents getting up and down while toileting?
18. Are grab bars and handrails mounted in the bathroom to support people of different heights?
19. Has toileting accessibility (e.g., toilet height, bedside location) been considered?
20. Does the current bed afford low height positions and braking?
21. Are there unnecessary restraints in furniture selection (including the use of bilateral full-length bed rails)?
22. Does the furniture support independent mobility of residents?
23. Is there ergonomic design in furniture selection to reduce resident fatigue (e.g., adjustable heights, standing workstations)?
24. Are there fall risks from furniture/equipment where activities are performed within the home?
25. Have call, communication systems, and integrated technology been selected to minimize public noise?
26. Has noise been controlled through the design (e.g., material selection)?
27. Is there space for safety alert signage (e.g., fall risk, isolation precaution) by the resident room entrance and/or the resident bed?
28. Is there safe resident access to slippery environments?
29 Are there safe transitions between areas?
30 Is there wheelchair accessibility?
31. Are there appropriate counter heights for the particular resident?
32. Are toe kicks or additional knee clearance needed?
33. Are the exterior plantings and walkways safe?
34. Can windows be opened easily to reduce strain upon resident's shoulders?
35. Does proper wayfinding or reminder signage exist which will assist a resident with impaired cognitive function?
36. Are exterior pathways clear to means of transportation?
37. Do the driveway and adjacent sidewalk/s have a smooth, gradual transition?

In one embodiment, hotspot module 122 may identify certain features in the home and identify correlated hazards using data models 146 of known issues. In another embodiment, hotspot module 122 may utilize machine learning/artificial intelligence module 123 to identify hazards and suggestions for re-configurations/renovations of the residence to address those hazards. For example, in an embodiment a machine learning model may be trained using multiple types of data sets, including one or more of a residence type data set, a resident type data set and a known hazard data set using data from known prior situations to recognize hazards in the environment. Once trained, the trained machine learning model may be used to identify potential hazards in areas of the residence using the residence data and the 3D graphical model 129a, 129b as input. In some embodiments, programmatic image recognition/classification may be used together with, or separately from, the trained machine learning model to identify objects in the 3D graphical model as a means to identify hazards. In a further embodiment, the machine learning model may be trained with additional data sets including one or more of a past recommendations data set holding previous recommendations for specified detected conditions, a health information technology (HIT) data set holding information regarding available types of technology for various health conditions, a health insurance information data set holding information regarding insurance eligibility for various types of suggested hazard remedies, a contractor information data set holding information regarding contractors available to perform installation of suggested remedies, a vendor data set holding information about different vendors with items for sale that are associated with suggested re-configurations/renovations, and a legal data set holding information about building codes and other legal requirements for implementing the suggestions.

In an embodiment, machine learning/artificial intelligence module 123 may support other forms of artificial intelligence beyond machine learning to perform the functions described herein. For example, neural networks may be employed to perform several tasks for health platform 120. For example, in some embodiments, AI may be used as a means to identify paths of travel, identify objects within the 3D graphical model, and/or identify product associations and proper accessible-design codes, standards and dimensioning.

Captured data 128 is used to create 3D graphical model 129a, 129b. 3D graphical model 129a, 129b provides an accurate digital twin view of the captured environment. Hotspot module 122 may generate indicators of identified hazards and related information within 3D graphical models 129a, 129b thus adding additional virtual information into the captured view and creating 3D AR model 130. This 3D AR model 130 may also include the suggested re-configurations/renovations either as a graphical indicator listing the suggestions or as a graphical depiction of the suggestion integrated into the captured 3D view (e.g. a new sink type displayed within 3D AR model). In one embodiment, the indicators of hazards or suggestions may include embedded links to additional information and resources that when selected display additional information. For example, such additional resources may include the International Code Council providing information regarding installations that are ADA-compliant. In one embodiment, 3D AR model 130 may be displayed via app 152 on mobile device 150. In another embodiment, 3D AR model 130 may be displayed via the online portal provided by health platform 120.

The suggested re-configurations/renovations may involve the movement of objects within the captured environment and/or the addition or removal of objects within the captured environment. Depending on resident age, ensuring the ability of the resident to perform Basic and Instrumental ADLs may take on increased importance when forming suggestions for re-configuration/renovations. For example, suggestions relating to toilets, bathing, medical management and lighting may all be dictated by ADLs appropriate for a particular resident.

Basic ADLs include:
Walking, or otherwise getting around the home or outside (i.e. ambulating).
Feeding-being able to get food from a plate into one's mouth.
Dressing and grooming-selecting clothes, putting them on, and adequately managing one's personal appearance.
Toileting-getting to and from the toilet, using it appropriately, and cleaning oneself.
Bathing-washing one's face and body in the bath or shower.
Transferring-being able to move from one body position to another. This includes being able to move from a bed to a chair, or into a wheelchair. This can also include the ability to stand up from a bed or chair in order to grasp a walker or other assistive device.

Instrumental ADLs include:
Managing finances, such as paying bills and managing financial assets.
Managing transportation, either via driving or by organizing other means of transport.
Shopping and meal preparation. This covers activities required to get a meal on the table. It also covers shopping for clothing and other items required for daily life.
House Cleaning and home maintenance. This means cleaning kitchens after eating, keeping one's living space reasonably clean and tidy, and keeping up with home maintenance.
Managing communication, such as the telephone and mail.
Managing medications, which covers obtaining medications and taking them as directed.

Continuing with the discussion of FIG. 1, health platform 120 may provide access to a network-accessible billboard 123. Billboard 124 may hold a variety of information including information about hazards, contractors, supplies and insurance posted by seniors or others. Billboard 124 may also hold information seniors post about themselves thereby providing a social meeting tool. In an embodiment, health platform 120 may provide access to reference materials 124. For example, reference materials 125 may relate to building, insurance, safety or other issues. Health platform 120 may also provide third party links 126, such as via a web page with listed third party links to contractors, supplies, insurance forms, etc.

Server 110 may include or provide access to database 140 holding information which may be used by hotspot module 122. Database 140 may include safety data 141 identifying known types of hazards. Database 140 may also include re-configuration data 142 providing known re-configurations for known hazards. Other types of data may also be included, including without limitation, legal data 143, building code data 144 and insurance data 145. Insurance data 145 may indicate which suggested renovations may be eligible for insurance reimbursement and may be used to determine which re-configurations of the senior environment should be suggested by health platform 120.

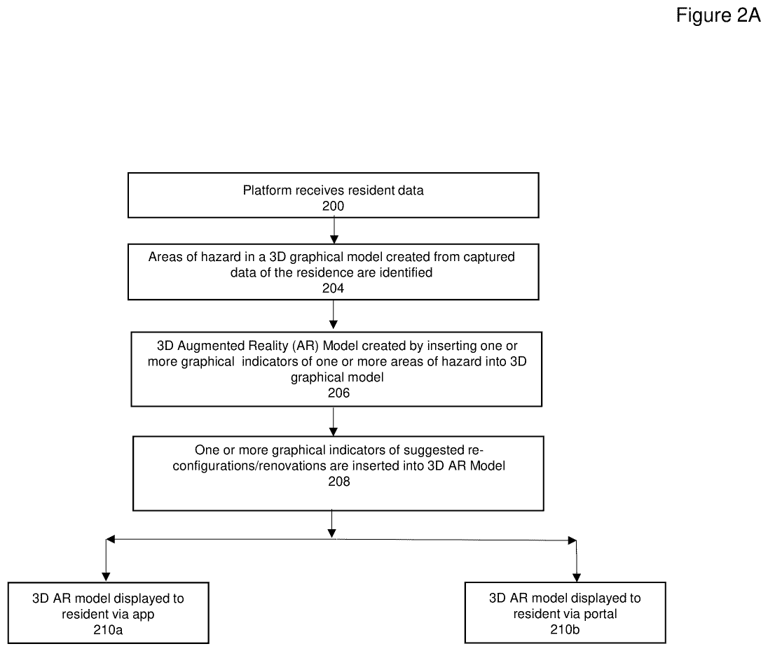

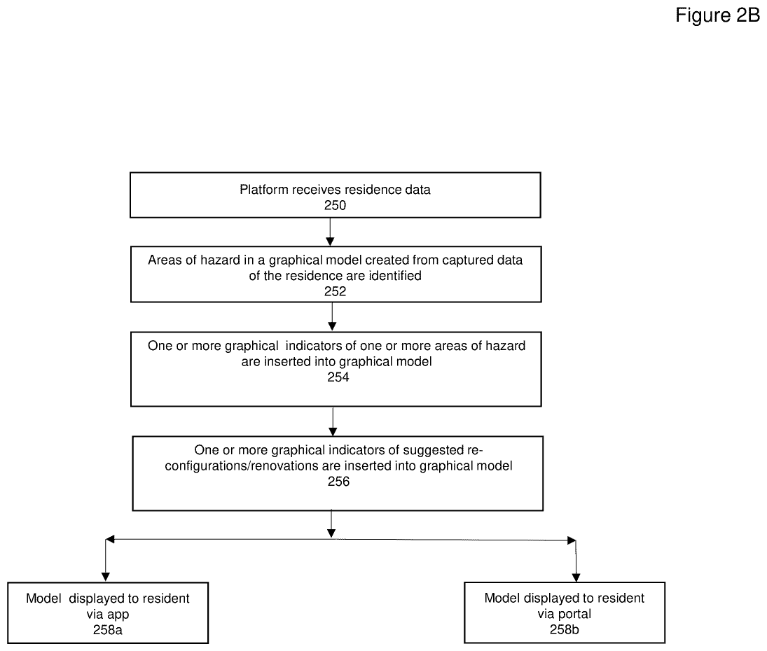

FIG. 2A depicts an exemplary sequence of steps performed for identifying and proposing remedies for hazards in a residence in an exemplary embodiment utilizing residence information and a received 3D graphical model created from captured data of the residence. The sequence begins with health platform 120 receiving residence data 127 (step 202). As noted previously, this residence data includes both information about the residence and information about its resident(s). Hotspot module 122 then identifies areas of hazard in a 3D graphical model 129a, 129b created from captured scan or image data of the residence (step 204). The 3D graphical model may be created by health platform 120 or received from a 3^{rd} party entity that created the model. In the scenario where health platform 120 creates 3D graphical model 129a, it first receives captured data 128 (captured scan or image data of a residence) and rendering module 121 creates 3D graphical model 129 from the captured data. It will be appreciated that the particular technique used to create 3D graphical model 129a, 129b will depend upon and vary based on the type of scan or image data that is captured but the result is the creation of a 3D graphical model that can be displayed as an immersive 3D graphical view that accurately captures the resident's environment. Of note, areas identified as hazardous may be specific to a particular resident's physical, cognitive function or acuity, as what is a hazard to a resident in one condition at a certain age may not be a hazard to another resident with a different physical or means of cognitive functioning at a different age. The hotspot module 122 creates a 3D AR model 130 by inserting one or more graphical indicators of identified hazards into the 3D graphical model (step 206). The hotspot module 122 also inserts one or more graphical indicators and/or graphical depictions of suggested re-configurations/renovations into 3D AR model 130 (step 208). The 3D AR model is then displayed via app 152 (step 210a) or via portal 210b.

In an alternative embodiment, instead of displaying the identified hotspots and suggestions in an immersive 3D view, the health platform uses a 2D rendering created from the captured data to display areas of hazard and possible suggestions for addressing those hazards to enhance resident safety. FIG. 2B depicts an exemplary sequence of steps performed for identifying and proposing remedies for hazards in a residence in an exemplary embodiment utilizing residence information and a 2D graphical model created from captured data of the residence. The sequence begins with health platform 120 receiving residence data 127 (step 250). As noted previously, this residence data includes both information about the residence and information about its residents. Hotspot module 122 identifies areas of hazard in a 2D graphical model that has been created from captured data of the residence (step 252). The hotspot module 122 inserts one or more graphical indicators of hazards into the graphical model (step 254). The hotspot module 122 also inserts one or more graphical indicators and/or graphical depictions of suggested re-configurations/renovations into the graphical model (step 256). The 2D graphical model is then displayed via app 152 (step 258a) or via portal 258b.

In some embodiments, hotspot module 122 examines measurements in the 3D graphical model data to help identify potential hazards in a residence. These measurements may be examined and considered in combination with residence data 127. As should be appreciated, some potential hazards may be hazards to everyone and others may be hazards to the particular resident or residents based on the information in the received residence data regarding physical and/or cognitive conditions or other information specific to the resident(s).

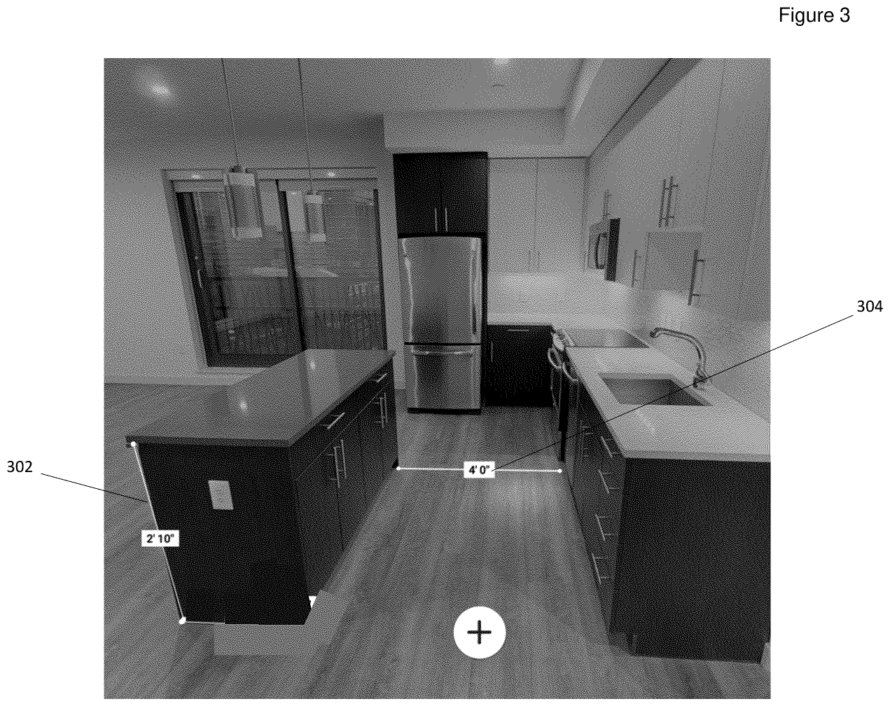

FIG. 3 depicts exemplary measurements in a 3D graphical model examined by a hotspot module to identify potential hazards in a residence. As non-limiting examples, hotspot module 122 may examine the data for 3D graphical model 129a, 129b to identify a counter height measurement 302 and clearance measurement 104 to determine their suitability for a resident who uses a wheelchair in the residence. It should be appreciated that the manner in which the measurements are identified by hotspot module 122 will vary based upon the manner in which the data was captured and used to create the 3D graphical model. For example, the act of scanning an environment with LIDAR will result in distances between captured objects being available in the LIDAR data to hotspot module 122. These measurements may then be compared to pre-determined criteria to detect for example a danger based upon previous known hazards and for code compliance in the construction of the residence.

In one embodiment, through human and blended AI analysis of the captured model data, the health platform identifies the presence of wayfinding and mobility hazards through trained visual image recognition.

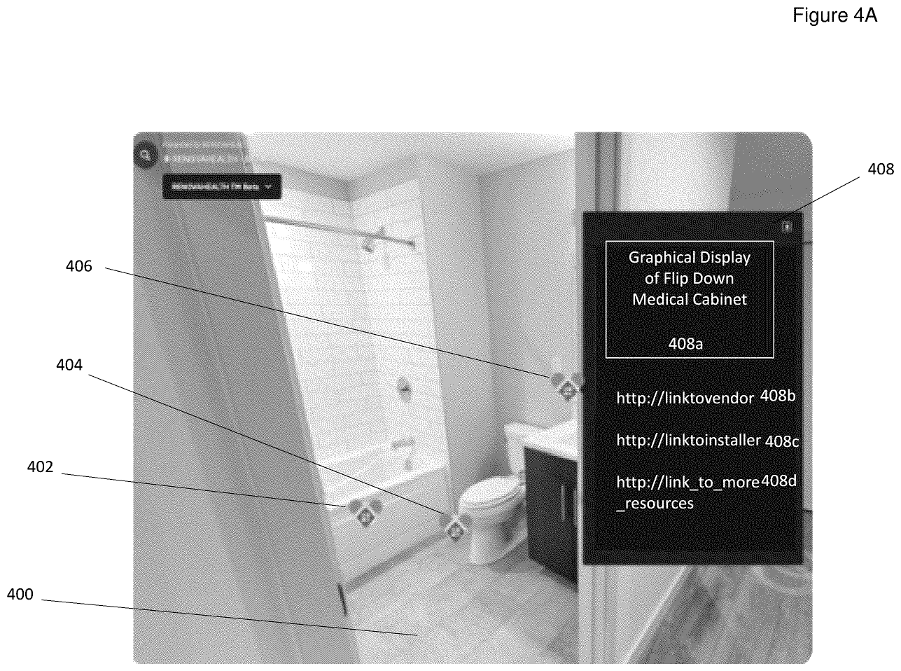

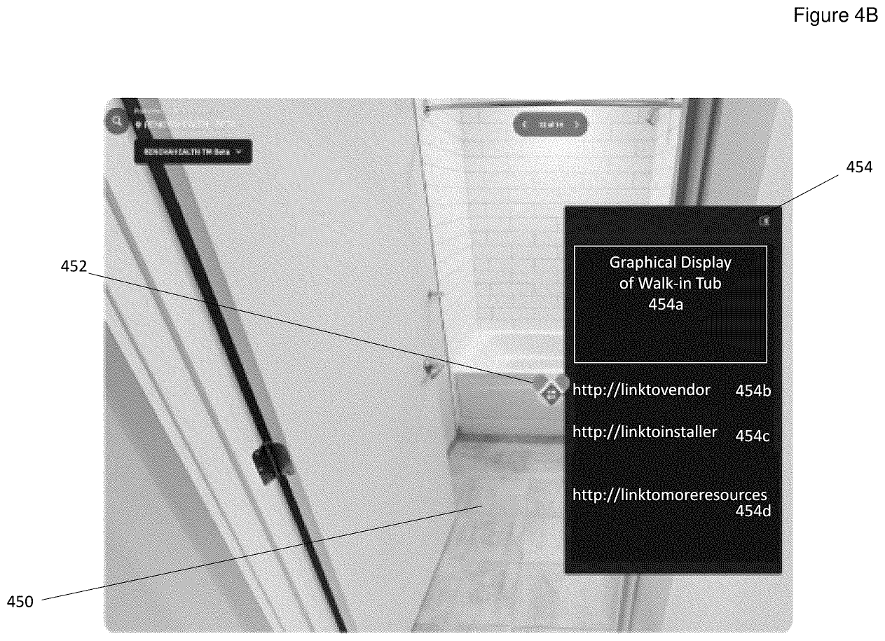

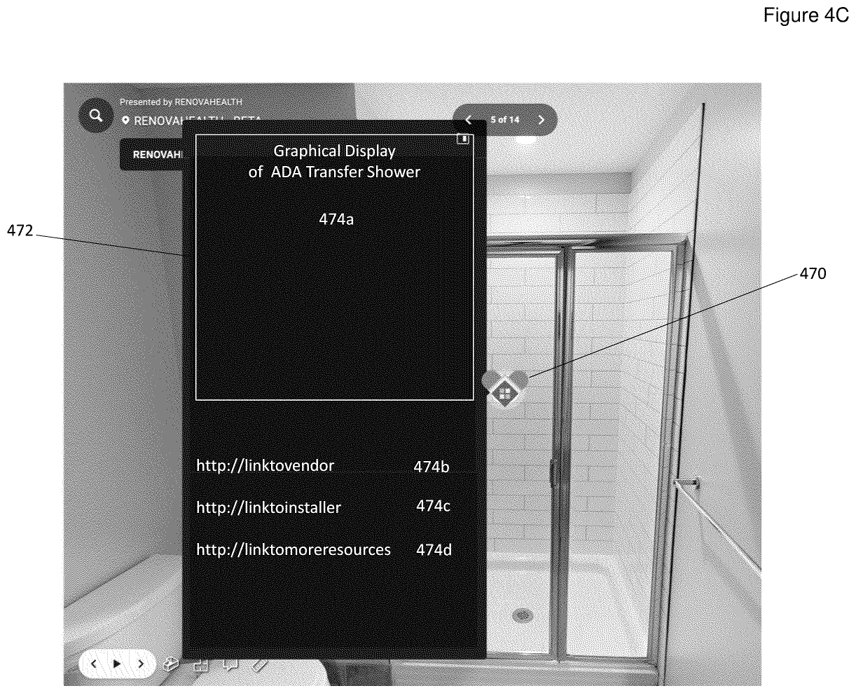

FIGS. 4A-4C depict 3D AR models of a residence that include inserted graphical hotspot indicators of potential hazards and associated displayed indicators of graphical indicators of suggested re-configurations/renovations of the residence in exemplary embodiments. For example, FIG. 4A depicts an exemplary 3D AR model of a residence in which hotspot indicators 402, 404 and 406 have been inserted into a 3D graphical view of a bathroom 400. The hotspot indicators indicate potential hazards for a tub (hotspot indicator 402), a toilet 404 (hotspot indicator 404) and a medicine cabinet (hotspot indicator 406). As previously noted, ‘hazard’ as used herein may refer not only to a dangerous condition but also to an object or condition that may represent an impediment to use of the residence by an elderly resident even if not dangerous per se. For example, the bathtub hotspot 402 may be present because the fixed tub lip represents a fall hazard when entering and exiting the tub. The toilet hotspot 404 may be marked as a hazard because of a toilet height is unsafe for those of limited mobility but also because it lacks an easy to operate mechanical flush plate. The medicine cabinet hotspot 406 may be marked as a hazard because it is inconvenient and/or dangerous for some residents to access based on their physical condition such as if the particular resident is in a wheelchair. Each of these inserted hotspots include embedded information that when accessed, such as being hovered over with a cursor or selected in some manner via either app or portal, displays additional information. For example, hotspot 406 includes graphically indicated suggestion 408 for re-configuration/renovation of a replacement to the current medicine chest with a flip down medicine cabinet which tilts down to provide access to a resident in a wheelchair. Graphically indicated suggestion 408 includes a displayed picture of the flip down medicine chest 408a, a link to a vendor 408b who sells the item, a link to an installer 408c and a link to further resources related to the suggestion 408d. For example, each of the suggested re-configuration/renovations may include or provide access to further resources regarding the suggestion such as information regarding its eligibility for insurance coverage based on the resident's insurance coverage information previously received by the platform, etc. The suggestion(s) provided by hotspot module 122 may also be based on information such as ADA and building code compliance.

Similarly, FIG. 4B depicts an exemplary 3D AR model of bathroom 450 in which a hotspot 452 indicates a potential tripping hazard with the bathtub. Graphically indicated suggestion 454 in this case is a walk-in tub in which the tub has a door into the tub which greatly reduces the potential tripping hazard. Graphically indicated suggestion 454 includes a displayed picture of the tub 454a, a link to a vendor 454b who sells the tub, a link to an installer 454c and a link to further resources related to the suggestion 454d.

Likewise, FIG. 4C depicts an exemplary 3D AR model of bathroom 470 in which hotspots 471 and 472 indicate a potential hazard with entry into the shower and provide code-compliant suggestions for residents to identify, understand, and rectify the space concern. Graphically indicated suggestion 474 in this example is an ADA-compliant shower without a fixed barrier into the shower which reduces the potential for falls when accessing the shower as the door doesn't need to be manipulated. Graphically indicated suggestion 474 includes a displayed picture of the shower 474a, a link to a vendor 474b who sells the shower, a link to an installer 474c and a link to further resources related to the suggestion 474d. It will be appreciated that the suggestions provided by embodiments may be formed in part by adherence to International Code Council A117.1 standards, codes, diagrams and dimensions as outlined under Federal law and regulation, where applicable.

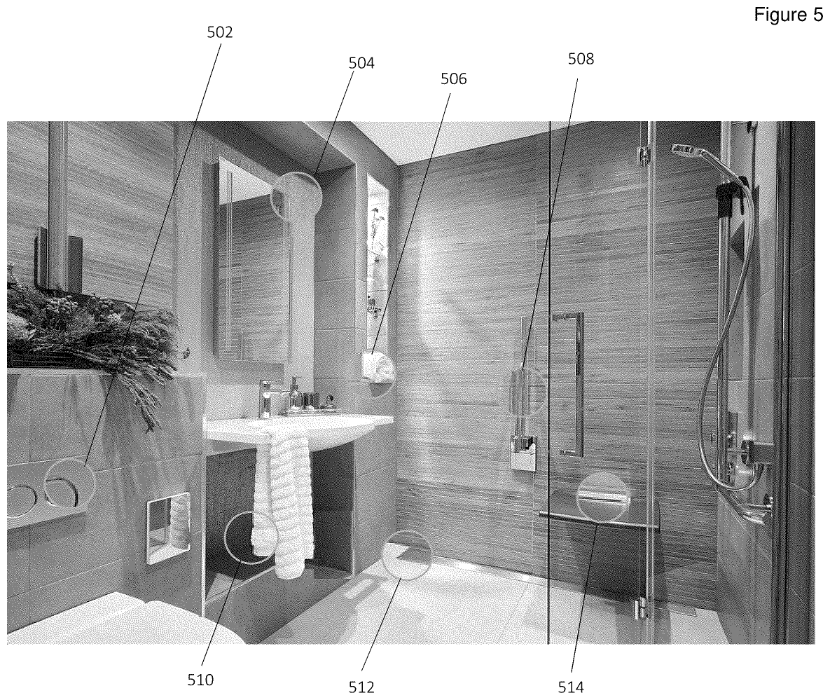

Instead of just providing an overlaid pop-up of the suggestion as shown in FIGS. 4A-4C, the suggested renovations/re-configurations may also be graphically integrated into the previously captured view of the residence to show what a portion of the residence would look like if the suggestion is followed and the current layout is updated. FIG. 5 depicts displays of suggested re-configurations/renovations integrated into a 3D graphical model of a bathroom 500 in an exemplary embodiment. The displayed suggestions include a push versus lever flushing system 502, integrated mirror lighting for the aging eye 504, flush, recessed wall cabinetry for ease of use and reach 506, folding grab bars for case of use and space restrictions 508, clearance beneath wall-mounted sink with integrated storage shelving 510, slot drain at back wall to reduce risk of slipping 512, and wall-mounted shower seat 514. As non-limiting examples, such re-configurations/renovations may include without limitation discreet shower seats which fold neatly up against the wall. Further suggestions may be comfort lever mixer taps and illuminated mirrors with demister pads. Additional suggestions may include comfort height toilets with easy-to-operate mechanical flush plates, low surface temperature electric towel rails and push-button call alarms (which are a more discrete, user-friendly, and hygienic alternative to traditional emergency red pull-cords). An additional suggestion may include slip-resistant porcelain floor tiles.

It will be appreciated that the suggestions provided by health platform 120 to allow a senior to age in place depend on the particular environment and the particular physical condition of the senior. For example, some suggestions that may be appropriate for some seniors but not others include no area throw rugs and/or the installation of guardrails or ramps at entrances. For some seniors it may make sense to suggest the installation of grab bars near the toilet and in the tub or shower. For some seniors the use of barrier-free shower entries, non-slip strips or non-skid mats on wood floors or surfaces that get wet may be appropriate. For other seniors, light switches at the top and bottom of stairs and the use of night lights may be advantageous. It will be appreciated that many additional types of suggestions are also within the scope of the present invention.

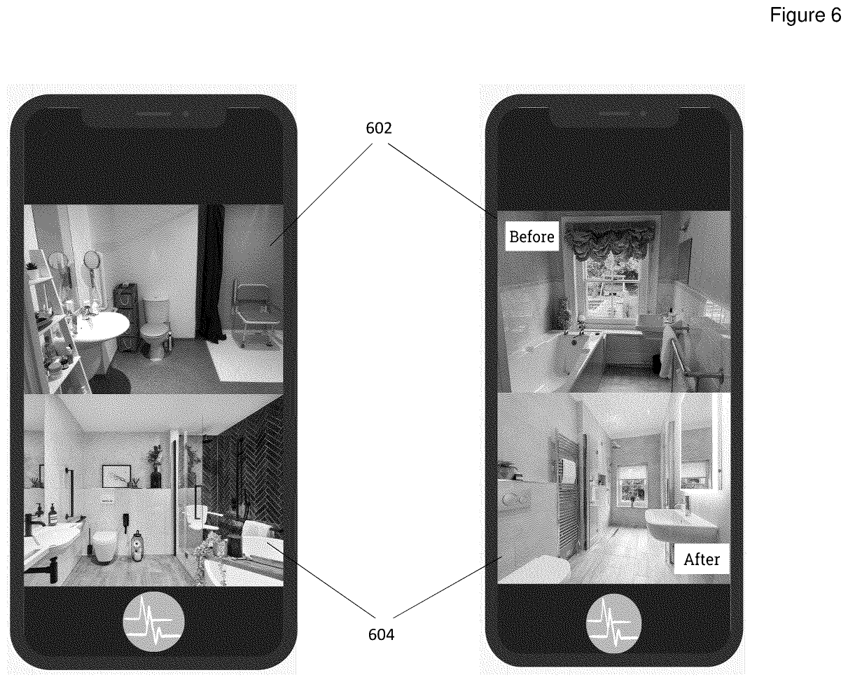

FIG. 6 depicts before and after renderings 602, 604 of bathroom renovations/re-configurations suggested by health platform 120 displayed by app 152 on a user's smartphone in an exemplary embodiment. On the left example, updated flooring, sinks, toilets, and a shower are depicted amongst other changes. On the right example, re-configuration of the room includes movement of the sink and tub away from the window, new floors, and other changes. It will be appreciated that many other suggested re-configuration/renovations in addition to those shown are within the scope of the present invention.

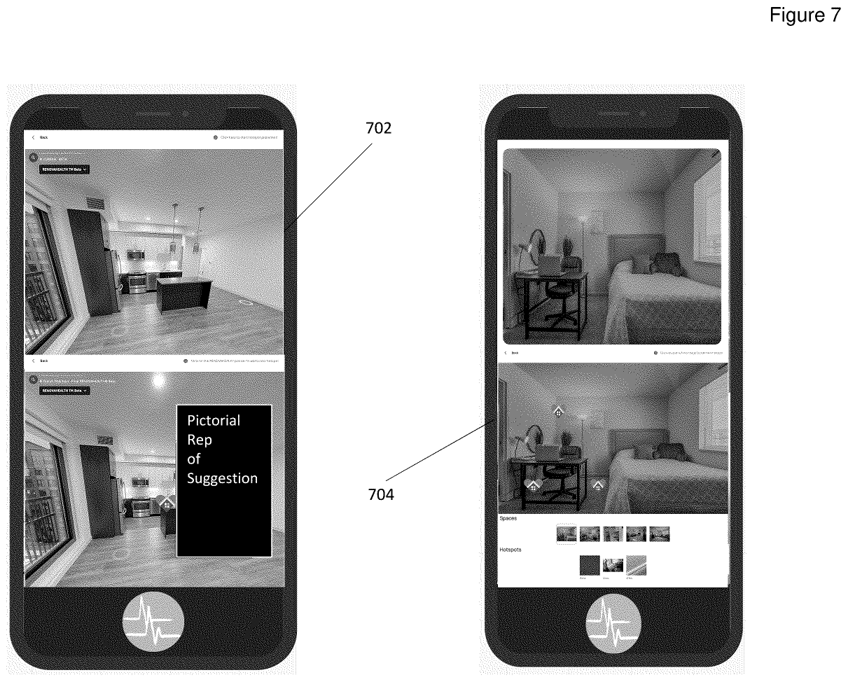

As noted above, the captured residence environment may be analyzed and hotspots presented in either 3D or 2D form. For example, FIG. 7 depicts before and after renderings of a 3D captured environment 702 displayed via a mobile device on the left and a 2D captured environment 704 displayed via a mobile device on the right. Both renderings 702, 704, display hotspot areas with potential hazards identified which could benefit from suggested re-configurations/renovations of the areas. As previously discussed, the suggestions may take many forms. In one embodiment, the suggestions may be for the use of digital health electronics and wearables for the older adult aging in place. In one embodiment the suggestions may involve the use of Extended Reality (XR) which may use sensor input from digital health electronics and/or wearables to form the suggestion and may include the use of a fully virtual reality (VR) presentation. In some embodiments, the captured environment may include advanced care settings such as Memory Care (MC) and Skilled Nursing Facilities (SNFs), and/or academic use integration in the setting of University Based Retirement Communities (UBRCs). It will be appreciated that many other suggested re-configuration/renovations in addition to those shown are within the scope of the present invention.

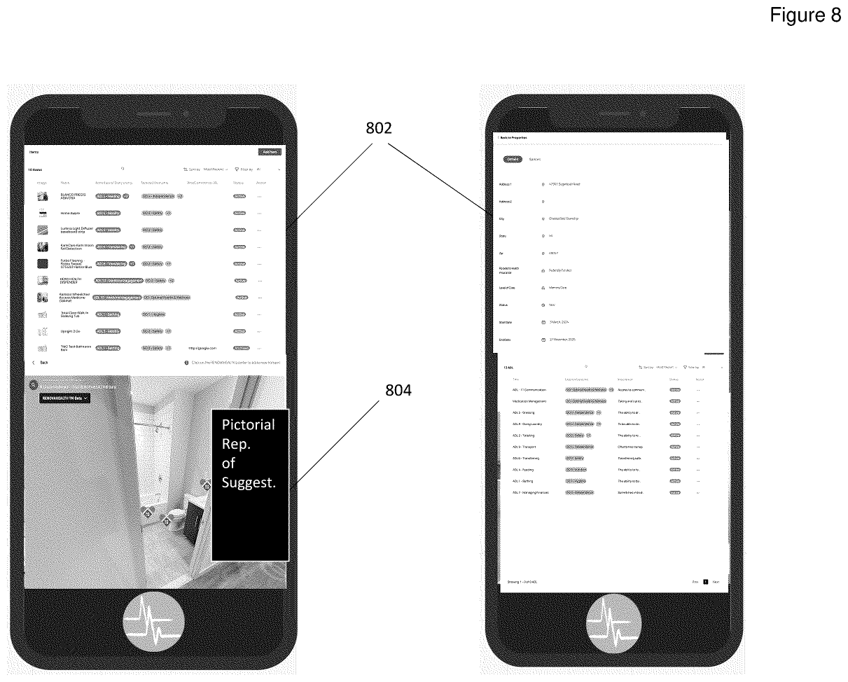

In some embodiments, the health platform described herein may generate and display information related to its suggestions via a dashboard type presentation. FIG. 8 depicts an exemplary dashboard 802 of suggestions indicating items listed by Activities of Daily Living and Desired Outcomes, along with a pictorial item representation 804 within the 3D captured residence environment suggesting products and resources to assist the older adult aging in place. It will be appreciated that many other suggested re-configuration/renovations in addition to those shown are within the scope of the present invention.

Portions or all of the embodiments of the present invention may be provided as one or more computer-readable programs or code embodied on or in one or more non-transitory mediums. The mediums may be, but are not limited to a hard disk, a compact disc, a digital versatile disc, ROM, PROM, EPROM, EEPROM, Flash memory, a RAM, or a magnetic tape. In general, the computer-readable programs or code may be implemented in any computing language.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described, the order of the acts may be modified in other implementations consistent with the principles of the invention. Further, non-dependent acts may be performed in parallel.

We claim:

1. A non-transitory medium holding computing device-executable instructions for a digital health platform, the instructions when executed causing at least one computing device equipped with one or more processors to:
receive over a network residence data, the residence data being data associated with a residence and at least one resident of the residence;
identify one or more areas of hazard in a 3D graphical model created from captured data, the captured data being one or more of scan data or image data of the residence;
create a 3D Augmented Reality (AR) model by inserting an indicator for each of the one or more identified areas of hazard into the 3D graphical model;
insert one or more graphical indicators of suggested re-configurations/renovations of the residence into the 3D AR model; and
provide a display of the 3D AR model with the suggested re-configurations/renovations of the residence.

2. The medium of claim 1, wherein the instructions when executed further cause the at least one computing device to:
depict graphically the suggested re-configurations/renovations in the 3D graphical model.

3. The medium of claim 1 wherein the graphical indicators of suggested re-configurations/renovations include embedded information regarding associated activities of daily living and related desired outcomes.

4. The medium of claim 1, wherein the instructions when executed further cause the at least one computing device to:
receive over the network the 3D graphical model.

5. The medium of claim 1, wherein the instructions when executed further cause the at least one computing device to:
receive over the network the captured data, the captured data being one or more of scan data or image data of the residence;
create the 3D graphical model of at least a portion of the residence using the captured data.

6. The medium of claim 1, wherein the instructions when executed further cause the at least one computing device to:
provide a mobile app, the mobile app configured to transmit the residence data to the digital health platform and to receive the suggested reconfigurations/renovations.

7. The medium of claim 1, wherein the instructions when executed further cause the at least one computing device to:
train a machine learning model to identify hazards in a residence, and
identify the one or more hazards in areas of the residence using the residence data and the 3D graphical model as input to the trained machine learning model.

8. The medium of claim 7 wherein the machine learning model is trained using a plurality of data sets including a residence type data set, a resident type data set and a known hazard data set.

9. The medium of claim 7 wherein the machine learning model is further trained to provide suggested re-configurations/renovations of the residence, and provides suggestions for re-configurations/renovations based on identifying the one or more hazards.

10. The medium of claim 9 wherein the plurality of data sets used to train the machine learning model further includes one or more of a past recommendations data set, a health information technology (HIT) data set, a health insurance information data set, a contractor information data set, a vendor data set and a legal data set.

11. The medium of claim 1 wherein programmatic image recognition of the 3D graphical model is performed to identify one or more of objects in the residence and the one or more hazards.

12. The medium of claim 1 wherein the suggested re-configurations/renovations involve one or more of the suggested removal, addition or movement of objects in a portion of the residence.

13. The medium of claim 1 wherein the suggested re-configurations/renovations are accompanied by additional information associated with the suggested re-configurations/renovations, the additional information including one or more links to one or more of materials, vendors of materials, contractors, insurance information and legal requirement information.

14. The medium of claim 1, wherein the instructions when executed further cause the at least one computing device to:
identify, using a machine learning/artificial intelligence module one or more paths of travel, objects within the 3D graphical model, product associations for e-commerce and residence-suitable design codes, standards and dimensions.

15. A non-transitory medium holding computing device-executable instructions for a digital health platform, the instructions when executed causing at least one computing device equipped with one or more processors to:
receive over a network residence data, the residence data being data associated with a residence and at least one resident of the residence;
identify one or more areas of hazard in a graphical model created from captured data, the captured data being one or more of scan data or image data of the residence;
insert an indicator for each of the one or more identified areas of hazard into the graphical model;
insert one or more graphical indicators of suggested re-configurations/renovations of the residence into the graphical model; and
provide a display of the graphical model with the suggested re-configurations/renovations of the residence.

16. The medium of claim 15, wherein the instructions when executed further cause the at least one computing device to:
depict graphically the suggested re-configurations/renovations in the graphical model.

17. A computing device-implemented method for providing a digital health platform via one or more computing devices, the one or more computing devices each equipped with one or more processors, the method comprising:
receiving over a network residence data, the residence data being data associated with a residence and at least one resident of the residence;
identifying one or more areas of hazard in a 3D graphical model created from captured data, the captured data being one or more of scan data or image data of the residence;
creating a 3D Augmented Reality (AR) model by inserting an indicator for each of the one or more identified areas of hazard into the 3D graphical model;
inserting one or more graphical indicators of suggested re-configurations/renovations of the residence into the 3D AR model; and
providing a display of the 3D AR model with the suggested re-configurations/renovations of the residence.

18. The method of claim 1, further comprising:
depicting graphically the suggested re-configurations/renovations in the 3D graphical model.

19. The method of claim 17 wherein the graphical indicators of suggested re-configurations/renovations include embedded information regarding associated activities of daily living and related desired outcomes.

20. The method of claim 17, further comprising:
receiving over the network the 3D graphical model.

21. The method of claim 17, further comprising:
receiving over the network the captured data, the captured data being one or more of scan data or image data of the residence;
creating the 3D graphical model of at least a portion of the residence using the captured data.

22. The method of claim 17, further comprising:
providing a mobile app, the mobile app configured to transmit the residence data to the digital health platform and to receive the suggested reconfigurations/renovations.

23. The method of claim 17, further comprising:
training a machine learning model to identify hazards in a residence, and
identifying the one or more hazards in areas of the residence using the residence data and the 3D graphical model as input to the trained machine learning model.

24. The method of claim 23 wherein the machine learning model is trained using a plurality of data sets including a residence type data set, a resident type data set and a known hazard data set.

25. The method of claim 23 wherein the machine learning model is further trained to provide suggested re-configurations/renovations of the residence, and provides suggestions for re-configurations/renovations based on identifying the one or more hazards.

26. The method of claim 25 wherein the plurality of data sets used to train the machine learning model further includes one or more of a past recommendations data set, a health information technology (HIT) data set, a health insurance information data set, a contractor information data set, a vendor data set and a legal data set.

27. The method of claim 17 wherein programmatic image recognition of the 3D graphical model is performed to identify one or more of objects in the residence and the one or more hazards.

28. The method of claim 17 wherein the suggested re-configurations/renovations involve one or more of the suggested removal, addition or movement of objects in a portion of the residence.

29. The method of claim 17 wherein the suggested re-configurations/renovations are accompanied by additional information associated with the suggested re-configurations/renovations, the additional information including one or more links to one or more of materials, vendors of materials, contractors, insurance information and legal requirement information.

30. The method of claim 17, further comprising:
identifying, using a machine learning/artificial intelligence module one or more paths of travel, objects within the 3D graphical model, product associations for e-commerce and residence-suitable design codes, standards and dimensions.

31. A computing device-implemented method for providing a digital health platform via one or more computing devices, the one or more computing devices each equipped with one or more processors, the method comprising:
receiving over a network residence data, the residence data being data associated with a residence and at least one resident of the residence;
identifying one or more areas of hazard in a graphical model created from captured data, the captured data being one or more of scan data or image data of the residence;
inserting an indicator for each of the one or more identified areas of hazard into the graphical model;
inserting one or more graphical indicators of suggested re-configurations/renovations of the residence into the graphical model; and
providing a display of the graphical model with the suggested re-configurations/renovations of the residence.

32. The method of claim 31, further comprising:
depicting graphically the suggested re-configurations/renovations in the graphical model.

33. A system for a digital health platform providing remote residence hazard analysis, comprising:
one or more computing devices equipped with one or more processors configured to provide a network-accessible digital health platform, the digital health platform including:
a network interface configured to receive residence data, the residence data being data associated with a residence and at least one resident of the residence;
a hotspot module configured to:
identify one or more areas of hazard in a 3D graphical model created from captured data, the captured data being one or more of scan data or image data of the residence,
create a 3D Augmented Reality (AR) model by inserting an indicator for each of the one or more identified areas of hazard into the 3D graphical model, and
insert one or more graphical indicators of suggested re-configurations/renovations of the residence into the 3D AR model; and
one or more of a network portal providing access to a display of the 3D AR model with the suggested re-configurations/renovations of the residence and a downloadable app configured to provide a display of the 3D AR model with the suggested re-configurations/renovations of the residence.

34. The system of claim 33, wherein the digital health platform further comprises:
a machine learning module:
trained to identify hazards in a residence using a plurality of data sets including a residence type data set, a resident type data set and a known hazard data set;
trained to provide suggested re-configurations/renovations of the residence based on identifying the one or more hazards using a plurality of data sets including one or more of a past recommendations data set, a health information technology (HIT) data set, a health insurance information data set, a contractor information data set, a vendor data set and a legal data set,
wherein the trained machine learning module identifies hazards in the residence and provides suggested re-configurations/renovations of the residence based on identifying the one or more hazards.
